(19) United States
(12) Patent Application Publication
(10) Pub. No.: US 2013/0289362 A1
(43) Pub. Date: Oct. 31, 2013

(54) INFRARED GUIDE STARS FOR ENDOSCOPIC ORIENTEERING

(71) Applicants: Lewis Kruglick, Poway, CA (US); Ezekiel Kruglick, Poway, CA (US)

(72) Inventors: Lewis Kruglick, Poway, CA (US); Ezekiel Kruglick, Poway, CA (US)

(73) Assignee: Empire Technology Development LLC, Wilmington, DE (US)

(21) Appl. No.: 13/819,673

(22) PCT Filed: Apr. 30, 2012
(86) PCT No.: PCT/US12/35905

§ 371 Date: Feb. 27, 2013

(51) Int. Cl. A61B 5/00

(52) U.S. Cl. CPC A61B 5/0059
USPC 600/301; 600/424

(57) ABSTRACT

Briefly stated, technologies are generally described for tracking a location and an orientation of a medical tool inside a body. The method may include positioning multiple light emitting diodes (LEDs) in infrared (IR) wavelength range near a tip of the medical tool. Upon insertion of the medical tool inside the body, the LEDs may emit IR light in unique patterns, which may be detectable through bodily tissue. The emitted light patterns may be detected through an IR detector device external to the body. One or more IR markers may be placed near the body to serve as reference points and the IR detector may determine the location and the orientation of the medical tool inside the body based on the detected light patterns. The location and the orientation of the medical tool inside the body may be displayed on a monitor.

### BACKGROUND

Unless otherwise indicated herein, the materials described in this section are not prior art to the claims in this application and are not admitted to be prior art by inclusion in this section.

During endoscopic procedures, such as upper gastro intestinal endoscopies and colonoscopies, a physician may insert a long flexible surgical instrument such as an endoscope to view and examine internal organs, to perform procedures, and take tissue samples. A light and a visual feedback device may be attached to the endoscope to provide visual feedback of the internal organs, so that the physician can identify an approximate location of the instrument within the body in order to navigate around the organs and to perform complex surgical and/or observation procedures.

In some cases, the endoscope may be a rigid instrument which may enable fairly simple position and location determination of the tools based on line of sight determinations from the handle of the tool to fiducials on the end of the rigid instrument in order to perform offset localization by extrapolating the location and orientation of the tip of the instrument based on the handle location. Some procedures, however, may require the use of flexible endoscopes and surgical instruments, and it can be difficult to determine the location and orientation of the endoscope within the body when the endoscope is flexible and a line of sight to the end of the endoscope is not available.

### SUMMARY

The present disclosure generally describes techniques for determining the location of a medical tool within a body based on relative locations of LEDs placed on the end of the medical tool and detected by a capture device external to the body. According to some examples, the present disclosure describes a method for tracking a location of a medical tool inside a body. The method may include positioning a plurality of light emitting diodes (LEDs) in infrared (IR) wavelength range near a tip of the medical tool, upon insertion of the medical tool inside the body, causing the LEDs to emit IR light with each LED emitting according to a predefined pattern, detecting the emitted light patterns through an IR detector device external to the body, and determining a location and an orientation of the medical tool inside the body based on the detected light patterns.

According to other examples, the present disclosure also describes a computing device for controlling a location tracking system of a medical tool inside a body. The computing device may include a memory configured to store instructions, and a processing unit configured to execute a location tracking application in conjunction with the instructions. The location tracking application may be configured to: upon insertion of the medical tool inside the body, cause a plurality of light emitting diodes (LEDs) in infrared (IR) wavelength range positioned near a tip of the medical tool to emit IR light with each LED configured to emit according to a predefined pattern, detect the emitted light patterns through an IR detector device external to the body, and determine a location and an orientation of the medical tool inside the body based on the detected light patterns.

According to further examples, the present disclosure may also describe a computer-readable storage medium having instructions stored thereon for tracking a location of a medical tool inside a body. The instructions may include positioning a plurality of light emitting diodes (LEDs), in infrared (IR) wavelength range near a tip of the medical tool, upon insertion of the medical tool inside the body, causing the LEDs to emit IR light with each LED emitting according to a predefined pattern, detecting the emitted light patterns through an IR detector device external to the body, and determining a location and a orientation of the medical tool inside the body based on the detected light patterns.

According to yet other examples, the present disclosure may further describe a system for location tracking of a medical tool inside a body. The system may include an endoscopic device with a plurality of light emitting diodes (LEDs) in infrared (IR) wavelength range positioned near a tip of the endoscopic device, a detection device for capturing IR light emitted from at least one of the LEDs outside the body, and a controller which may be configured to upon insertion of the endoscopic device inside the body, cause the LEDs to emit the IR light with each LED emitting according to a predefined pattern, receive detected IR light information from the detector device, and determine a location and an orientation of the endoscopic device inside the body based on the detected light patterns.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 illustrates an example endoscopic procedure setup, where an endoscope is employed to examine internal organs;

FIG. 2 illustrates an example infrared detection based location and orientation determination of a flexible endoscopic tool;

FIG. 3 illustrates an example endoscopic tool and placement of infrared light (IR) emitting diodes (LEDs) on the endoscopic tool for location and orientation determination;

FIG. 4 illustrates an example graph of a change in absorption coefficient for fat tissue with varying light wavelength enabling selection of emission ranges for LEDs to be used in an endoscopic tool;

FIG. 5 illustrates a general purpose computing device, which may be used to control a system implementing infrared detection based location and orientation determination of an endoscopic tool;

FIG. 6 is a flow diagram illustrating an example method that may be performed by a computing device such as device 500 in FIG. 5; and

FIG. 7 illustrates a block diagram of an example computer program product, all arranged in accordance with at least some embodiments described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to methods, apparatus, systems, devices, and/or computer program products related to determining location of a medical tool within a body based on relative locations of LEDs placed on the end of the medical tool and detected by a capture device external to the body.

Briefly stated, technologies are generally described for tracking a location of a medical tool inside a body. The method may include positioning multiple light emitting diodes (LEDs) in infrared (IR) wavelength range near a tip of the medical tool. Upon insertion of the medical tool inside the body, the LEDs may emit IR light in unique patterns, which may be detectable through bodily tissue. The emitted light patterns may be detected through an IR detector device external to the body. One or more IR markers may be placed near or attached to the body to serve as reference points and the IR detector may determine the location and orientation of the medical tool inside the body based on the detected light patterns. The location and orientation of the medical tool inside the body may be displayed on a monitor.

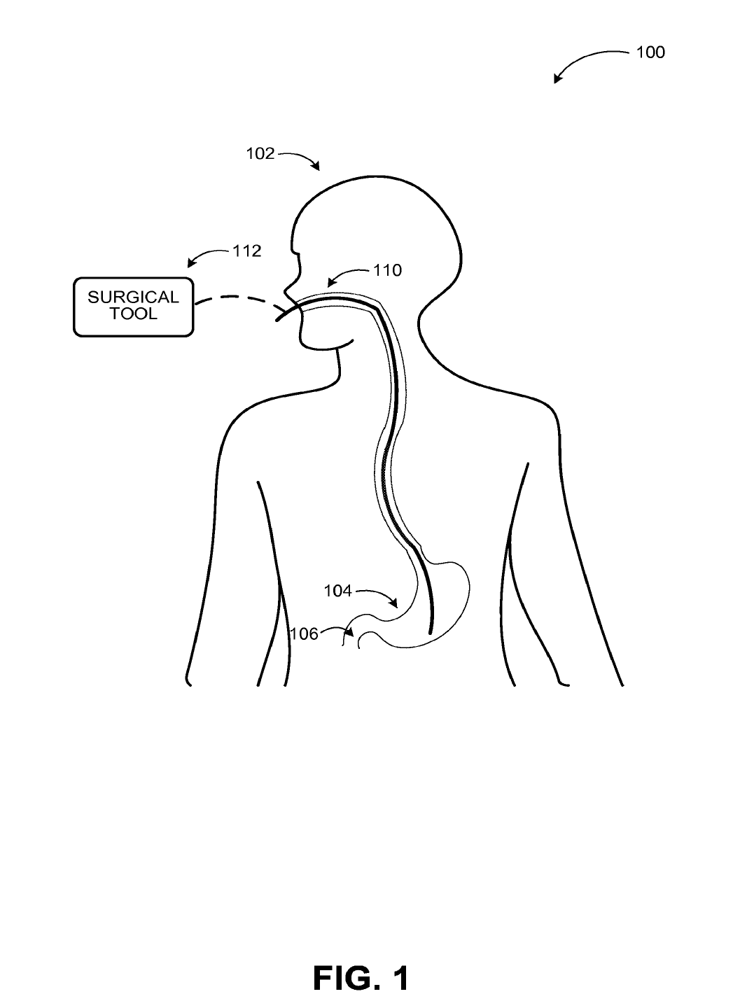

FIG. 1 illustrates an example endoscopic procedure setup, where an endoscope is employed to examine internal organs, arranged in accordance with at least some embodiments described herein. An example endoscopic procedure is a procedure enabling a physician to insert a long flexible tube, such as an endoscope 112, into the body 102 in order to view internal organs and tissues, to take tissue samples, and to perform various surgical procedures. Endoscopes may be inserted into the body via natural bodily orifices, such as the mouth, ears, and rectum, and during minimally invasive surgical procedures, an endoscope may be inserted through small incisions.

An example endoscopic procedure as demonstrated in diagram 100, may be an upper endoscopy, a procedure that allows the physician to look at the inside of the esophagus 110, stomach 104 and duodenum 106. A thin, flexible, lighted tube, or the endoscope 112 may be guided into the mouth and throat, then into the esophagus 110, stomach 104 and duodenum 106. The endoscope 112 may allow the physician to view the inside of this area of the body, as well as to insert instruments through a scope for the removal of a sample of tissue for biopsy.

Some other example endoscopic procedures which may use a endoscope for enabling the surgeon to view the internal body may include enteroscopy, esophagogastroduodenoscopy, colonoscopy, sigmoidoscopy, endoscopic retrograde cholangiopancreatography, duodenoscope-assisted cholangiopancreatoscopy, anoscopy, proctoscopy, rectoscopy, bronchoscopy, otoscopy, cystoscopy, gynoscopy, colposcopy, hysteroscopy, falloposcopy, laparoscopy, arthroscopy, thoracoscopy, mediastinoscopy, amnioscopy, fetoscopy, panendoscopy, epiduroscopy, and apicoectomy. The procedures listed above are not intended to be exhaustive, but are illustrative of some example procedures during which a surgeon may use a scope.

In an example embodiment, the endoscope 112 may be a rigid or flexible tube, and may include a light delivery system to illuminate the internal organs under inspection and a visual feedback device for providing visual feedback of the internal organs and the surgical tools to the surgeon. The visual feedback from the camera may be provided on an external viewing monitor for enabling the surgeon to view the enlarged organs and surgical tools which may be inserted inside the body with the endoscope 112 in order to perform procedures. It is important to track the location of the endoscope 112 to know exactly where in the body the endoscope 112 is located and how it is oriented, so that the surgeon may know what the endoscope 112 and the surgical tools would touch if they are moved or extended. This can be difficult when the endoscope 112 is a flexible tool and may be navigating through complex tissue and organs within the body.

Some existing localization systems may determine a location of the endoscope 112 based on relating the location to fixed reference points, or fiducials, on the rigid endoscope 112. For example, these systems may use a camera as a location determination device outside the body and flashing LED lights placed on the handle of the rigid endoscope for identifying the location of the endoscope 112 within the body. These example systems may require direct line of sight from the camera to the LEDs on the handle that are rigidly related to the working tip of the endoscope in order to perform offset localization, extrapolating the location of the tip of the endoscope 112 based on the handle location. This method, however, is incompatible with flexible surgical tools and endoscopes, as the reference point to the surgical handle is not fixed when a flexible tool or endoscope is used.

A system according to embodiments may provide enable determination of a location and orientation of a flexible medical tool, such as an endoscope while the scope is inside the body. The example system may be an infrared detection based system which may use long-wave infrared laser emitting diodes placed near the end of the endoscope and may localize them using flash encoding and optical reference localization.

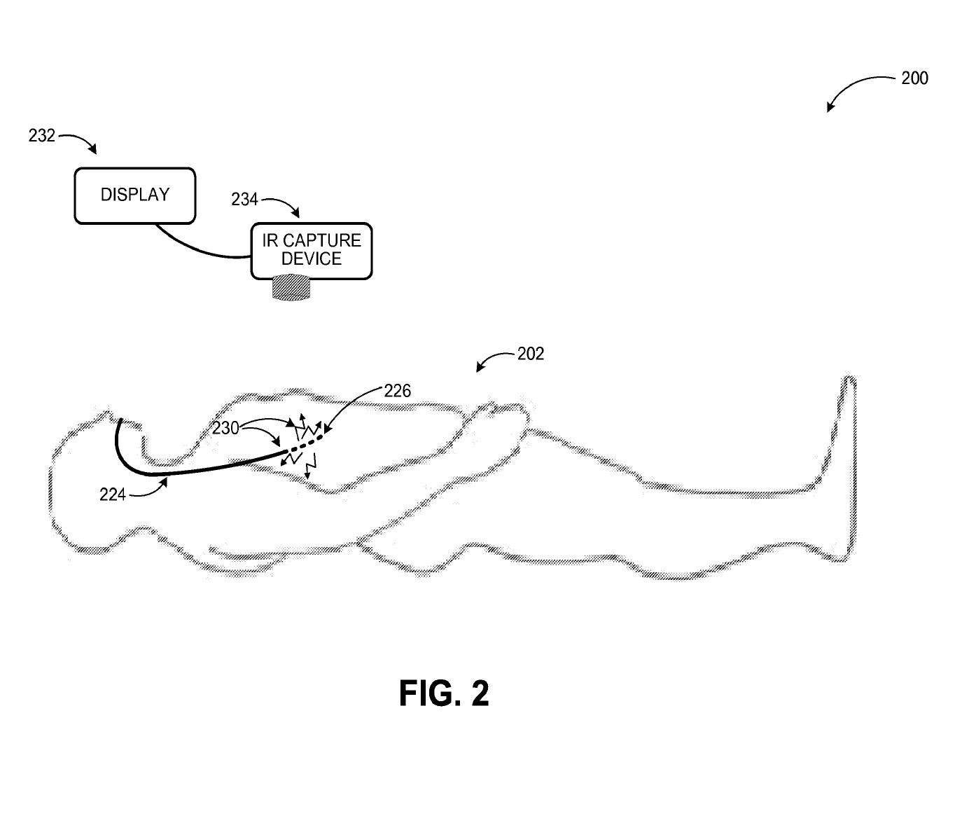

FIG. 2 illustrates an example infrared detection based location and orientation determination of a flexible endoscopic tool, arranged in accordance with at least some embodiments described herein. As demonstrated in diagram 200, infrared (IR) laser emitting diodes (LEDs) may be placed near the end of the flexible tool or endoscope 224, which may enable the location and the orientation of the endoscope 224 within the body 202 to be determined based on the relative locations of the LEDs.

In a system according to embodiments a plurality of light emitting diodes (LEDs) or laser diodes may be positioned near the tip of the flexible endoscope 224. The LEDs may emit infrared (IR) light, which may be in a wavelength range enabling the infrared light to penetrate the flesh, such that the IR light may be detectable outside the body 202 by an IR capture device 234. For example, a wavelength emission range of from about 700 nm to about 870 nm may be IR wavelengths that enable the IR wavelength to penetrate tissue in the body including flesh, fat, and some internal organs. The wavelength and/or emission level may be adjustable such that the IR light can be customized based on the patient's anatomy including varying fat content of the patient, the location of the endoscope 224 in the body and varying tissues and fat present in the location. Additionally, the wavelength and/or emission level may be adjusted based on the sensitivity of the IR capture device.

The IR capture device 234 may be configured to determine the location and orientation of the endoscope 224 based on the detected IR light 230. In an example embodiment, the LEDs may be placed around the tip 226 of the endoscope 224 in positions such that at least two LEDS may be visible from any angle. For example, at least three LEDs may be placed around the circumference of the tip 226 of the endoscope 224 in equal distances from each other such that at least two are visible from any angle as the endoscope 224 is rotated within the body 202. Additionally, a second set of at least three LEDs may be placed on the outside surface of the endoscope 224, at a distance of at least one wavelength away from the tip 226 of the endoscope. Each LED in the second set of LEDs may also be positioned in equal distances from each other such that at least two LEDs are visible from any angle as the endoscope 224 is translated and rotated within the body 202. Further, each LED may be configured to flash a distinctive IR light 230 pattern such that the IR capture device 234 may individually identify and distinguish each LED on the endoscope. The LEDs may use a wide angle lens such that the IR light 230 pattern emitted by each LED is viewable from a wide array of angle, thus increasing the visibility and detection of the IR light 230 by the IR capture device 234. Upon insertion of the endoscope 224 inside the body 202, each of the LEDs may emit the IR light 230 with each LED emitting its distinctive pattern. The IR capture device 234 may detect the emitted light patterns and may determine the location and orientation of the endoscope 224 based on the detected light patterns.

In a system according to embodiments, the location and orientation of the endoscope 224 emitting the IR light 230 patterns from the LEDs may be determined based on a relative position to IR markers, which may be placed on predefined positions on or near the body 202. For example, IR markers may be placed as reference points near a specific known location on the body, such as a joint or bone. Additionally, since the IR markers may emit IR light in a wavelength which may penetrate tissue, including bone, flesh, fat and organs, the IR markers may be placed beneath the body 202, which may allow uninterrupted access to the front of the body 202 during surgical procedures. The IR capture device 234 may determine the three dimensional position of each LED on the tip 226 of the endoscope 224 relative to one or more of the IR marker reference points based on the distinctive IR light 230 pattern of each LED. Since the LED lights may be placed around circumference of the tip 226 of the endoscope, the three dimensional position may also include a rotational position such that that surgeon may be able to determine how the endoscope is rotated in the body as well as where the endoscope is precisely located. The IR capture device 234 may display the determined location and orientation of the endoscope 224 on a display monitor 232 coupled with the IR capture device 234 external to the body for enabling the surgeon to continuously view the location of the endoscope 224 while the endoscope 224 is inside the body 202.

In an additional embodiment, the IR capture device 234 may utilize an Indium Antimonide charge coupled detection device for detecting the IR light 230 as the IR light 230 is transmitted from the LEDs through the body 202. The Indium Antimonide charge coupled detection device may provide IR wavelength detection up to 5.3 um wavelengths. Further, because of the wavelengths of the IR light 230 emitted by the LEDs, a microbolometer array may be used to detect the emitted IR light 230. The use of a microbolometer array, for example, may allow not only the detection of the LEDs within the body, but also may enable mapping of body thermal emissions for simultaneous measurement of blood flow and pooling within the body.

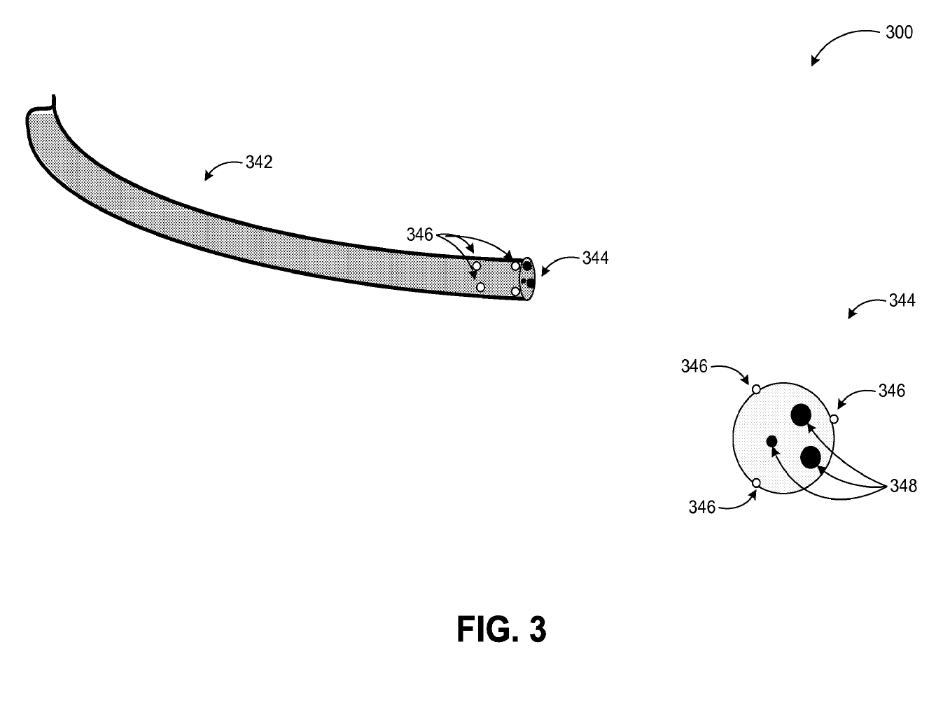

FIG. 3 illustrates an example endoscopic tool and placement of infrared light (IR) emitting diodes (LEDs) on the endoscopic tool for location and orientation determination, arranged in accordance with at least some embodiments described herein. As previously described, the IR capture device may be configured to determine the location and orientation of the endoscope 342 based on the detected IR light emitted from a plurality of LEDs 346, 344 placed near the tip of the endoscope. The plurality of LEDs 346, 344 may be positioned such that at least two LEDS may be visible from any angle, and may enable the three dimensional and rotational location and orientation of the endoscope to be determined.

In an example embodiment, a first set of LEDs 348 may be placed around circumference of the tip 344 of the endoscope 342 in positions such that at least two of the first set of LEDS 348 may be visible from any angle. For example, at least three of the first set of LEDs 348 may be placed around the circumference of the tip 344 of the endoscope in equal distances from each other such that at least two are visible from any angle as the endoscope 342 is rotated within the body. Additionally, a second set of at least three LEDs 346 may be placed on the outside surface of the endoscope 342, at a distance of at least one wavelength away from the tip 344 of the endoscope 342. Each LED in the second set of LEDs 346 may also be positioned in equal distances from each other such that at least two of the LEDs from the second set of LEDs 346 are visible from any angle as the endoscope 342 is translated and rotated within the body.

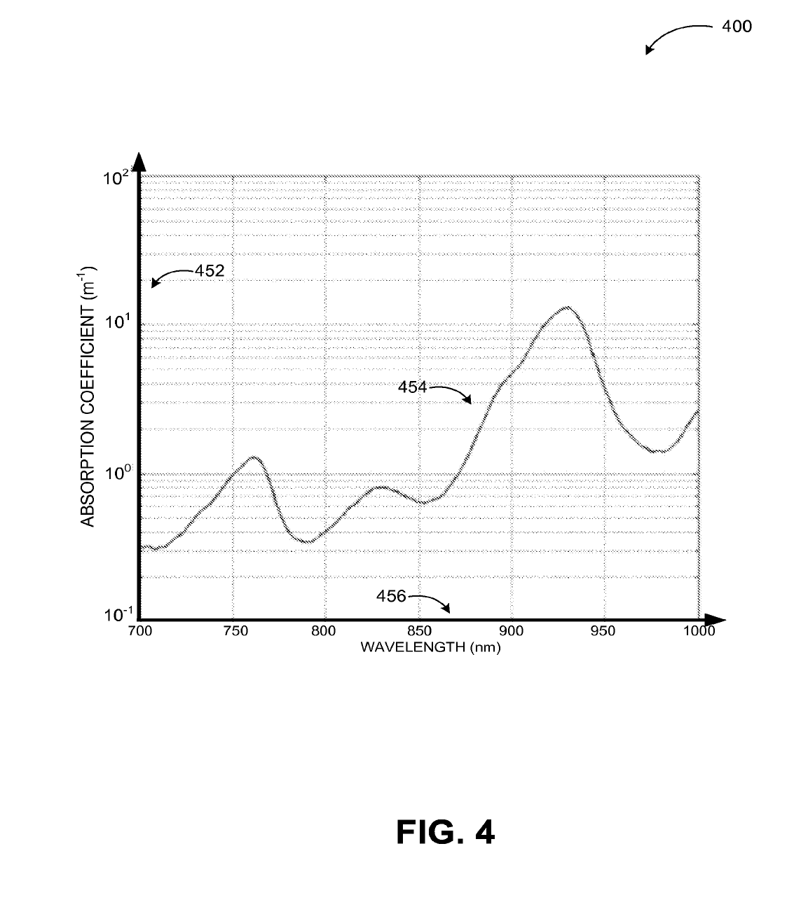

FIG. 4 illustrates an example graph of a change in absorption coefficient for fat tissue with varying light wavelength enabling selection of emission ranges for LEDs to be used in an endoscopic tool, arranged in accordance with at least some embodiments described herein. The LEDs positioned on the endoscope may emit infrared light, which may be in a wavelength range enabling the infrared light to penetrate body tissue, including the flesh, fat, and internal organs, such that the IR light may be detectable outside the body by an IR capture device.

As demonstrated in diagram 400, the absorption coefficient curve 454 for fat tissue across a wavelength range of 700 nm to 1000 nm may vary between 0.2 m⁻¹ and 15 m⁻¹. Thus, depending on the tissue type and depth, a suitable emission level and/or detector sensitivity may be selected for the emitted IR light to be detected outside of the body. Even at attenuations up to 80 dB, the IR light may be detectable by commercially available IR detectors. In some example embodiments, the IR wavelength may be selected in a range that provides lower absorption coefficients 452, for example from about 700 nm to about 870 nm, where the absorption coefficients for fat tissue are approximately 1 m⁻¹ or less.

Absorption spectra can be measured for additional body tissues including flesh, fat, and some internal organs and the preferred wavelength emission level may be adjustable such that the wavelength of the emitted IR light wavelength can be optimized to penetrate the bodily tissue of the patient. The IR light wavelength may be customized based on the patient's anatomy including varying fat content, and additionally based on the orientation of the medical tool in the body and varying tissues and fat present in the location. Additionally, the preferred wavelength emission level may be adjusted based on the sensitivity of the IR capture device.

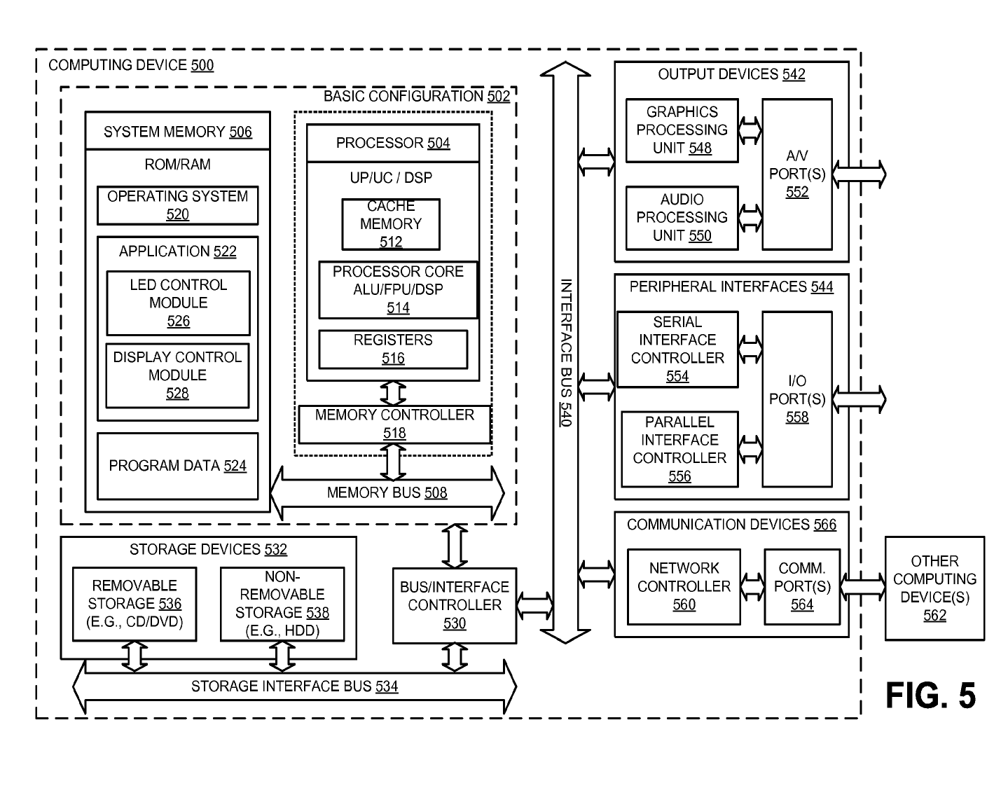

FIG. 5 illustrates a general purpose computing device, which may be used to control a system implementing infrared detection based location and orientation determination of an endoscopic tool, arranged in accordance with at least some embodiments described herein. In a very basic configuration 502, computing device 500 typically includes one or more processors 504 and a system memory 506. A memory bus 508 may be used for communicating between processor 504 and system memory 506.

Depending on the desired configuration, processor 504 may be of any type including but not limited to a microprocessor (μP), a microcontroller (μC), a digital signal processor (DSP), or any combination thereof. Processor 504 may include one more levels of caching, such as a level cache memory 512, a processor core 514, and registers 516. Example processor core 514 may include an arithmetic logic unit (ALU), a floating point unit (FPU), a digital signal processing core (DSP Core), or any combination thereof. An example memory controller 518 may also be used with processor 504, or in some implementations memory controller 518 may be an internal part of processor 504.

Depending on the desired configuration, system memory 506 may be of any type including but not limited to volatile memory (such as RAM), non-volatile memory (such as ROM, flash memory, etc.) or any combination thereof. System memory 506 may include an operating system 520, one or more applications 522, and program data 524. Application 522 may include an LED control module 526 that is arranged to configure LEDs positioned on an endoscope located within the body to emit IR light. The application 522 may also include a display control module 528 that is arranged to enable an IR capture device to detect the emitted IR light as the IR light is transmitted through a body. Program data 524 may include the emitted IR light data, and related orientation and position data. This data may be useful in determining the orientation of the endoscope based on the relative orientations of the LEDs on the endoscope. This described basic configuration 502 is illustrated in FIG. 5 by those components within the inner dashed line.

Computing device 500 may have additional features or functionality, and additional interfaces to facilitate communications between basic configuration 502 and any required devices and interfaces. For example, a bus/interface controller 530 may be used to facilitate communications between basic configuration 502 and one or more data storage devices 532 via a storage interface bus 534. Data storage devices 532 may be removable storage devices 536, non-removable storage devices 538, or a combination thereof. Examples of removable storage and non-removable storage devices include magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include volatile and nonvolatile, removable and non-removable media implemented in any method or technology for storage of information, such as computer readable instructions, data structures, program modules, or other data.

System memory 506, removable storage devices 536 and non-removable storage devices 538 are examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 500. Any such computer storage media may be part of computing device 500.

Computing device 500 may also include an interface bus 540 for facilitating communication from various interface devices (e.g., output devices 542, peripheral interfaces 544, and communication devices 546) to basic configuration 502 via bus/interface controller 530. Example output devices 542 include a graphics processing unit 548 and an audio processing unit 570, which may be configured to communicate to various external devices such as a display or speakers via one or more A/V ports 572. Example peripheral interfaces 544 include a serial interface controller 574 or a parallel interface controller 576, which may be configured to communicate with external devices such as input devices (e.g., keyboard, mouse, pen, voice input device, touch input device, etc.) or other peripheral devices (e.g., printer, scanner, etc.) via one or more I/O ports 578. An example communication device 546 includes a network controller 560, which may be arranged to facilitate communications with one or more other computing devices 562 over a network communication link via one or more communication ports 564.

The network communication link may be one example of a communication media. Communication media may typically be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media.

Computing device 500 may be implemented as a portion of a small-form factor portable (or mobile) electronic device such as a cell phone, a personal data assistant (PDA), a personal media player device, a wireless web-watch device, a personal headset device, an application specific device, or a hybrid device that include any of the above functions. Computing device 500 may also be implemented as a personal computer including both laptop computer and non-laptop computer configurations. Moreover computing device 500 may be implemented as a networked system or as part of a general purpose or specialized server.

Example embodiments may also include methods. These methods can be implemented in any number of ways, including the structures described herein. One such way is by machine operations, of devices of the type described in the present disclosure. Another optional way is for one or more of the individual operations of the methods to be performed in conjunction with one or more human operators performing some of the operations while other operations are performed by machines. These human operators need not be collocated with each other, but each can be only with a machine that performs a portion of the program. In other examples, the human interaction can be automated such as by pre-selected criteria that are machine automated.

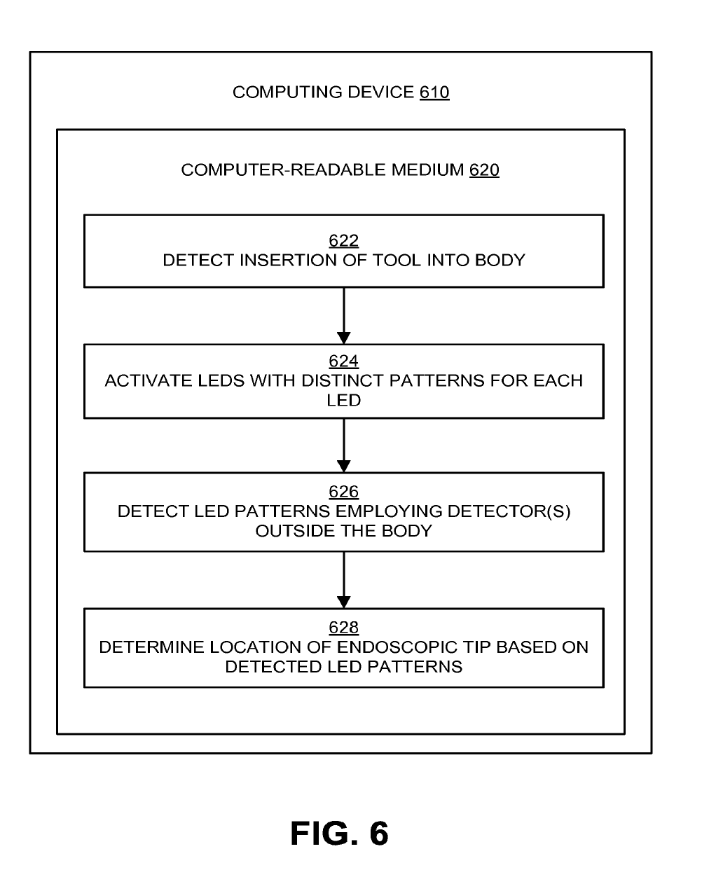

FIG. 6 is a flow diagram illustrating an example method that may be performed by a computing device such as computing device 500 in FIG. 5, arranged in accordance with at least some embodiments described herein. Example methods may include one or more operations, functions or actions as illustrated by one or more of blocks 622, 624, 626, 628 and/or 630. The operations described in blocks 622 through 630 may also be stored as computer-executable instructions in a computer-readable medium such as computer-readable medium 620 of computing device 610.

A process for determining the location and orientation of a medical tool within a body based on relative locations of LEDs placed on the end of the medical tool and detected by a capture device external to the body may begin with block 622, “DETECT INSERTION OF TOOL INTO BODY”. At block 622, a medical tool, such as an endoscope may be inserted into the body through a bodily orifice for viewing internal organs and tissues within a body. The endoscope may include a plurality of LEDs emitting IR light which may be positioned around the tip of the endoscope.

Block 622 may be followed by block 624, “ACTIVATE LEDS WITH DISTINCT PATTERS FOR EACH LED.” At block 624, the LEDs positioned on the tip of the endoscope may be configured to flash the IR light in distinct patterns such that each LED is identifiable and distinguishable by its unique IR light pattern.

Block 624 may be followed by block 626, “DETECT LED PATTERNS EMPLOYING DETECTORS OUTSIDE THE BODY.” At block 626, an IR capture device positioned external to the body may detect the emitted IR light from each LED. The IR light may be of a wavelength which enables the IR light to be transmitted through the bodily tissue such that the IR capture device may detect the light outside the body.

Block 626 may be followed by block 628, “DETERMINE LOCATION OF ENDOSCOPIC TIP BASED ON DETECTED LED PATTERNS.” At block 628, the IR capture device may determine the three dimensional and rotational location and orientation of each LED relative to IR marker reference points which may be placed on or near the body. Based on the relative locations of each of the LEDs on the tip of the endoscope, the precise three dimensional and rotational location and orientation of the endoscope within the body may be determined.

The blocks 622 through 628 may be performed by a dynamic modeling module of a processor of a first computing device (e.g. processor 504 or graphics processing unit 546 of a computing device 500), and may, on the other hand be performed by a graph matching module of the same processor or another processor on a second computing device coupled to the first computing device through a network. Of course, all blocks may be performed by a single module as well.

The blocks included in the above described process are for illustration purposes. Determining the location and orientation of a medical tool within a body based on relative locations of LEDs placed on the end of the medical tool and detected by a capture device external to the body may be performed by similar processes with fewer or additional blocks. In some examples, the blocks may be performed in a different order. In some other examples, various blocks may be eliminated. In still other examples, various blocks may be divided into additional blocks, or combined together into fewer blocks.

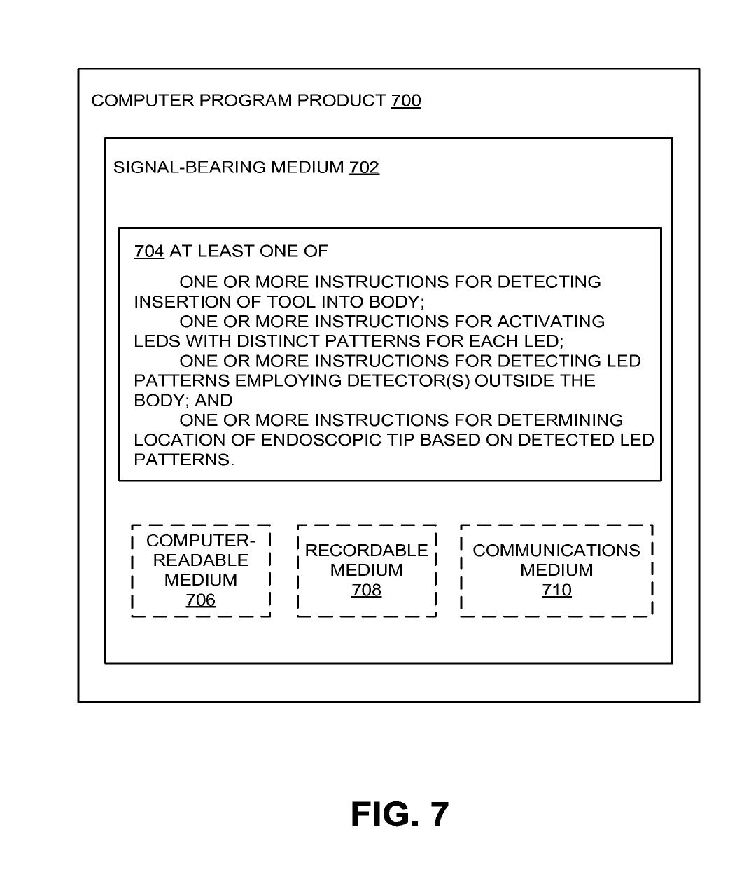

FIG. 7 illustrates a block diagram of an example computer program product, arranged in accordance with at least some embodiments described herein. In some examples, as shown in FIG. 7, computer program product 700 may include a signal bearing medium 702 that may also include machine readable instructions 704 that, when executed by, for example, a processor, may provide the functionality described above with respect to FIG. 5 and FIG. 6. Thus, for example, referring to processor 504, the display control module 528 may undertake one or more of the tasks shown in FIG. 5 in response to instructions 704 conveyed to processor 504 by medium 702 to perform actions associated with determining the location and orientation of a medical tool within the body based on the location of LEDs as described herein. Some of those instructions may include detecting insertion of the tool into the body, activate LEDs with distinct patterns for each LED, detecting LED patterns employing detector(s) outside the body, and determining the location and orientation of an endoscopic tip based on the detected LED patterns.

In some implementations, signal bearing medium 702 depicted in FIG. 7 may encompass a computer-readable medium 706, such as, but not limited to, a hard disk drive, a Compact Disc (CD), a Digital Versatile Disk (DVD), a digital tape, memory, etc. In some implementations, signal bearing medium 702 may encompass a recordable medium 708, such as, but not limited to, memory, read/write (R/W) CDs, R/W DVDs, etc. In some implementations, signal bearing medium 702 may encompass a communications medium 710, such as, but not limited to, a digital and/or an analog communication medium (e.g., a fiber optic cable, a waveguide, a wired communications link, a wireless communication link, etc.). Thus, for example, program product 700 may be conveyed to one or more modules of the processor 504 by an RF signal bearing medium 702, where the signal bearing medium 702 is conveyed by a wireless communications medium 710 (e.g., a wireless communications medium conforming with the IEEE 802.11 standard).

The present disclosure describes a method for tracking a location and orientation of a medical tool inside a body. The method may include positioning a plurality of light emitting diodes (LEDs) in infrared (IR) wavelength range near a tip of the medical tool, upon insertion of the medical tool inside the body, causing the LEDs to emit IR light with each LED emitting according to a predefined pattern, detecting the emitted light patterns through an IR detector device external to the body, and determining a location and an orientation of the medical tool inside the body based on the detected light patterns.

According to some examples, the method may include employing the medical tool for at least one from a set of: enteroscopy, esophagogastroduodenoscopy, colonoscopy, sigmoidoscopy, endoscopic retrograde cholangiopancreatography, duodenoscope-assisted cholangiopancreatoscopy, anoscopy, proctoscopy, rectoscopy, bronchoscopy, otoscopy, cystoscopy, gynoscopy, colposcopy, hysteroscopy, falloposcopy, laparoscopy, arthroscopy, thoracoscopy, mediastinoscopy, amnioscopy, fetoscopy, panendoscopy, epiduroscopy, and apicoectomy.

According to some examples, the method may include positioning a first set of at least three LEDs along a circumference of the tip of the medical tool, and positioning a second set of at least three LEDs on an outside surface of the medical tool about one wavelength away from the tip of the medical tool.

According to some examples, the first set of LEDs may be positioned at about equal distances from each other such that the emitted IR light is detectable from any angle around the body. The second set of LEDs may positioned at about equal distances from each other such that the emitted IR light is detectable from any angle around the body. The LEDs may include laser diodes, and the LEDs may have wide angle lenses.

According to some examples, the method may include selecting the LEDs in an emission range from about 700 nm to about 870 nm.

According to other examples, the method may include adjusting an emission power level of the LEDs based on one or more of a location of the medical tool, a fat content of the body, a sensitivity of a detector device capturing the emitted IR light.

According to other examples, the location of the medical tool may include a rotational position, and the medical tool may be a flexible tool. The predefined pattern may include causing each LED to flash employing at least one of a different frequency and emission period.

According to other examples, the method may include employing one of an indium antimonide charge coupled detection device and a microbolometer array to detect the emitted light pattern. The method may also include mapping of body thermal emissions for simultaneous measurement of blood flow and pooling employing a detector device used for detecting the emitted light pattern.

According to further examples, the method may include placing at least one IR marker on the body for spatial comparison with the detected light pattern. The method may also include displaying the location and the orientation of the medical tool inside the body on a monitor.

The present disclosure also describes a computing device for controlling a location tracking system of a medical tool inside a body. The computing device may include a memory configured to store instructions, and a processing unit configured to execute a location tracking application in conjunction with the instructions. The location tracking application may be configured to: upon insertion of the medical tool inside the body, cause a plurality of light emitting diodes (LEDs) in infrared (IR) wavelength range positioned near a tip of the medical tool to emit IR light with each LED configured to emit according to a predefined pattern, detect the emitted light patterns through an IR detector device external to the body, and determine a location and an orientation of the medical tool inside the body based on the detected light patterns.

According to some examples the medical tool may be employed for at least one from a set of: enteroscopy, esophagogastroduodenoscopy, colonoscopy, sigmoidoscopy, endoscopic retrograde cholangiopancreatography, duodenoscope-assisted cholangiopancreatoscopy, anoscopy, proctoscopy, rectoscopy, bronchoscopy, otoscopy, cystoscopy, gynoscopy, colposcopy, hysteroscopy, falloposcopy, laparoscopy, arthroscopy, thoracoscopy, mediastinoscopy, amnioscopy, fetoscopy, panendoscopy, epiduroscopy, and apicoectomy.

According to some examples, a first set of at least three LEDs may be positioned along a circumference of the tip of the medical tool and a second set of at least three LEDs are positioned on an outside surface of the medical tool about one wavelength away from the tip of the medical tool, and the first set of LEDs may be positioned at about equal distances from each other such that the emitted IR light is detectable from any angle around the body.

According to some examples, the second set of LEDs may be positioned at about equal distances from each other such that the emitted IR light is detectable from any angle around the body. The LEDs may include laser diodes, and the LEDs may have wide angle lenses.

According to other examples, the location tracking application may be further configured to adjust an emission power level of the LEDs based on one or more of a location of the medical tool, a fat content of the body, a sensitivity of a detector device capturing the emitted IR light.

According to other examples, the location of the medical tool may include a rotational position, and the medical tool may be a flexible tool. The predefined pattern may include causing each LED to flash employing at least one of a different frequency and emission period.

According to other examples, one of an indium antimonide charge coupled detection device and a microbolometer array may be employed to detect the emitted light pattern.

According to yet other examples, the location tracking application may be further configured to map of body thermal emissions for simultaneous measurement of blood flow and pooling employing a detector device used for detecting the emitted light pattern. At least one IR marker may be placed on the body for spatial comparison with the detected light pattern. The location tracking application may be further configured to display the location and the orientation of the medical tool inside the body on a monitor.

The present disclosure may also describe a computer-readable storage medium having instructions stored thereon for tracking a location of a medical tool inside a body. The instructions may include positioning a plurality of light emitting diodes (LEDs) in infrared (IR) wavelength range near a tip of the medical tool, upon insertion of the medical tool inside the body, causing the LEDs to emit IR light with each LED emitting according to a predefined pattern, detecting the emitted light patterns through an IR detector device external to the body, and determining a location and an orientation of the medical tool inside the body based on the detected light patterns.

According to some examples, the instructions may include employing the medical tool for at least one from a set of: enteroscopy, esophagogastroduodenoscopy, colonoscopy, sigmoidoscopy, endoscopic retrograde cholangiopancreatography, duodenoscope-assisted cholangiopancreatoscopy, anoscopy, proctoscopy, rectoscopy, bronchoscopy, otoscopy, cystoscopy, gynoscopy, colposcopy, hysteroscopy, falloposcopy, laparoscopy, arthroscopy, thoracoscopy, mediastinoscopy, amnioscopy, fetoscopy, panendoscopy, epiduroscopy, and apicoectomy.

According to some examples, the instructions may include positioning a first set of at least three LEDs along a circumference of the tip of the medical tool, and positioning a second set of at least three LEDs on an outside surface of the medical tool about one wavelength away from the tip of the medical tool.

According to some examples, the first set of LEDs may be positioned at about equal distances from each other such that the emitted IR light is detectable from any angle around the body. The second set of LEDs may be positioned at about equal distances from each other such that the emitted IR light is detectable from any angle around the body. The LEDs may include laser diodes, and the LEDs may have wide angle lenses.

According to some examples, the instructions may include selecting the LEDs in an emission range from about 700 nm to about 870 nm. The instructions may also include adjusting an emission power level of the LEDs based on one or more of a location of the medical tool, a fat content of the body, a sensitivity of a detector device capturing the emitted IR light.

According to other examples, the location of the medical tool may include a rotational position, and the medical tool may be a flexible tool. The predefined pattern may include causing each LED to flash employing at least one of a different frequency and emission period.

According to other examples, the instructions may include employing one of an indium antimonide charge coupled detection device and a microbolometer array to detect the emitted light pattern. The instructions may also include mapping of body thermal emissions for simultaneous measurement of blood flow and pooling employing a detector device used for detecting the emitted light pattern.

According to other examples, the instructions may include placing at least one IR marker on the body for spatial comparison with the detected light pattern. The instructions may further include displaying the location and the orientation of the medical tool inside the body on a monitor.

The present disclosure may further describe a system for location tracking of a medical tool inside a body. The system may include an endoscopic device with a plurality of light emitting diodes (LEDs) in infrared (IR) wavelength range positioned near a tip of the endoscopic device, a detection device for capturing IR light emitted from at least one of the LEDs outside the body, and a controller which may be configured to upon insertion of the endoscopic device inside the body, cause the LEDs to emit the IR light with each LED emitting according to a predefined pattern, receive detected IR light information from the detector device, and determine a location and an orientation of the endoscopic device inside the body based on the detected light patterns.

According to some examples, a first set of at least three LEDs may be positioned along a circumference of the tip of the endoscopic device and a second set of at least three LEDs are positioned along a circumference of the tip of the endoscopic device. The LEDs may have wide angle lenses such that the emitted IR light is detectable from any angle around the body.

According to some examples, the endoscopic device may be a flexible device with at least one of a monitoring device, a surgical device, a radiation therapy device, and a radio frequency (RF) ablation device affixed at the tip of the endoscopic device. The detection device may be one of an indium antimonide charge coupled detection device, and a microbolometer array to detect the emitted light pattern.

According to other examples, the system may include a display for presenting the location and the orientation of the medical tool inside the body on a monitor.

There is little distinction left between hardware and software implementations of aspects of systems; the use of hardware or software is generally (but not always, in that in certain contexts the choice between hardware and software may become significant) a design choice representing cost vs. efficiency tradeoffs. There are various vehicles by which processes and/or systems and/or other technologies described herein may be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples may be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, may be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Versatile Disk (DVD), a digital tape, a computer memory, a solid state drive, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein may be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity of systems; control motors for moving and/or adjusting components and/or quantities).

A typical data processing system may be implemented utilizing any suitable commercially available components, such as those typically found in data computing/communication and/or network computing/communication systems. The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermediate components. Likewise, any two components so associated may also be viewed as being “operably connected”, or “operably coupled”, to each other to achieve the desired functionality, and any two components capable of being so associated may also be viewed as being “operably couplable”, to each other to achieve the desired functionality. Specific examples of operably couplable include but are not limited to physically connectable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method for tracking a location of a medical tool inside a body, the method comprising:
positioning a plurality of light emitting diodes (LEDs) in infrared (IR) wavelength range near a tip of the medical tool by:
positioning a first set of at least three LEDs along a circumference of the tip of the medical tool, and
positioning a second set of at least three LEDs on an outside surface of the medical tool about one wavelength away from the tip of the medical tool;
upon insertion of the medical tool inside the body, causing the LEDs to emit IR light with each LED emitting according to a predefined pattern;
detecting the emitted light patterns through an IR detector device external to the body; and
determining a location and an orientation of the medical tool inside the body based on the detected light patterns.

2. The method according to claim 1, further comprising:
employing the medical tool for at least one from a set of: enteroscopy, esophagogastroduodenoscopy, colonoscopy, sigmoidoscopy, endoscopic retrograde cholangiopancreatography, duodenoscope-assisted cholangiopancreatoscopy, anoscopy, proctoscopy, rectoscopy, bronchoscopy, otoscopy, cystoscopy, gynoscopy, colposcopy, hysteroscopy, falloposcopy, laparoscopy, arthroscopy, thoracoscopy, mediastinoscopy, amnioscopy, fetoscopy, panendoscopy, epiduroscopy, and apicoectomy.

3. (canceled)

4. The method according to claim 1, wherein the first set of LEDs are positioned at about equal distances from each other such that the emitted IR light is detectable from any angle around the body.

5. The method according to claim 1, wherein the second set of LEDs are positioned at about equal distances from each other such that the emitted IR light is detectable from any angle around the body.

68. (canceled)

9. The method according to claim 1, further comprising:
adjusting an emission power level of the LEDs based on one or more of a location of the medical tool, a fat content of the body, a sensitivity of a detector device capturing the emitted IR light.

10. The method according to claim 1, wherein the location of the medical tool includes a rotational position.

1112. (canceled)

13. The method according to claim 1, further comprising:
employing one of a an indium antimonide charge coupled detection device and a microbolometer array to detect the emitted light pattern.

14. The method according to claim 13, further comprising:
mapping of body thermal emissions for simultaneous measurement of blood flow and pooling employing a detector device used for detecting the emitted light pattern.

1516. (canceled)

17. A computing device for controlling a location tracking system of a medical tool inside a body, the computing device comprising:
a memory configured to store instructions; and
a processing unit configured to execute a location tracking application in conjunction with the instructions, wherein the location tracking application is configured to:
upon insertion of the medical tool inside the body, cause a plurality of light emitting diodes (LEDs) in infrared (IR) wavelength range positioned near a tip of the medical tool to emit IR light with each LED configured to emit according to a predefined pattern, wherein a first set of at least three LEDs are positioned along a circumference of the tip of the medical tool and a second set of at least three LEDs are positioned on an outside surface of the medical tool about one wavelength away from the tip of the medical tool;
detect the emitted light patterns through an IR detector device external to the body; and
determine a location and an orientation of the medical tool inside the body based on the detected light patterns.

1821. (canceled)

22. The computing device according to claim 17, wherein the LEDs include laser diodes.

23. The computing device according to claim 17, wherein the LEDs have wide angle lenses.

2425. (canceled)

26. The computing device according to claim 17 wherein the medical tool is a flexible tool.

27. The computing device according to claim 17, wherein the predefined pattern includes causing each LED to flash employing at least one of a different frequency and emission period.

2829. (canceled)

30. The computing device according to claim 17, wherein at least one IR marker is placed on the body for spatial comparison with the detected light pattern.

31. The computing device according to claim 17, wherein the location tracking application is further configured to:
display the location and the orientation of the medical tool inside the body on a monitor.

3247. (canceled)

48. A system for location tracking of a medical tool inside a body, the system comprising:
an endoscopic device with a plurality of light emitting diodes (LEDs) in infrared (IR) wavelength range positioned near a tip of the endoscopic device, wherein a first set of at least three LEDs are positioned along a circumference of the tip of the endoscopic device and a second set of at least three LEDs are positioned on an outside surface of the medical tool about one wavelength away from the tip of the endoscopic device;
a detection device for capturing IR light emitted from at least one of the LEDs outside the body; and
a controller configured to:
upon insertion of the endoscopic device inside the body, cause the LEDs to emit the IR light with each LED emitting according to a predefined pattern;
receive detected IR light information from the detector device; and
determine a location and an orientation of the endoscopic device inside the body based on the detected light patterns.

49. (canceled)

50. The system according to claim 48, wherein the LEDs have wide angle lenses such that the emitted IR light is detectable from any angle around the body.

51. The system according to claim 48, wherein the endoscopic device is a flexible device with at least one of a monitoring device, a surgical device, a radiation therapy device, and a radio frequency (RF) ablation device affixed at the tip of the endoscopic device.

52. The system according to claim 48, wherein the detection device is one of an indium antimonide charge coupled detection device and a microbolometer array to detect the emitted light pattern.

53. The system according to claim 48, further comprising a display for presenting the location and the orientation of the medical tool inside the body on a monitor.
